(19) United States
(12) Patent Application Publication
(10) Pub. No.: US 2019/0015381 A1
(43) Pub. Date: Jan. 17, 2019

(54) Cannabinoid Formulation and Products

(71) Applicant: Angelina Fanous, Strongsville, OH (US)

(72) Inventor: Angelina Fanous, Strongsville, OH (US)

(21) Appl. No.: 16/033,434

(22) Filed: Jul. 12, 2018

Related U.S. Application Data
(60) Provisional application No. 62/531,656, filed on Jul. 12, 2017.

(51) Int. Cl. A61K 31/352

(52) U.S. Cl. CPC A61K 31/352; A61K 31/05; A61K 2300/00

(57) ABSTRACT

Methods, formulations and products are disclosed to treat and/or alleviate and/or otherwise improve the condition of a patient with certain medical conditions such as neuro-degenerative ailments, including certain cannabinoid formulations as described, e.g., in certain relative proportions to one another.

### RELATED APPLICATIONS

This application claims priority to U.S. Provisional Application No. 62/531,656, entitled “Cannabinoid Formulation and Products,” filed on Jul. 12, 2017, which is hereby incorporated by reference.

### TECHNICAL FIELD

The present application relates to compounds and products using cannabinoid formulations useful for enhancing the state or well-being of patients having certain medical conditions, and methods for making and using the same.

### BACKGROUND

Medical technology is making or seeking advances for treatment of medical conditions such as various neuro-degenerative diseases and conditions. Diagnosable examples of such conditions include multiple sclerosis (MS), amyotrophic lateral sclerosis (ALS), Parkinson's disease, and others.

In addition, the medical community, and the human community at large, seeks ways to enhance the state (mental and physical) of patients with neuro-degenerative conditions, and to improve their lives and reduce their suffering from these conditions. The cannabis flower, also known as marijuana, contains a number of cannabinoids that chemically or physiologically affect the neurotransmitters of the brain. For example, tetrahydrocannabinol (THC) is understood to have such an effect, which in proper application is credited with improving the wellbeing of patients suffering from a variety of symptoms.

The present disclosure is directed to some embodiments of methods, formulations and products made to achieve or assist in the treatment and management of symptoms of certain medical conditions.

### SUMMARY

An embodiment of this invention is directed to a therapeutic formulation of cannabinoids, which can be administered or used by inhaling said formulation combustion by-products, ingesting said formulation, injecting said formulation, or topically applying a spreadable product of said formulation. The formulation comprising a first cannabinoid comprising THC, a second cannabinoid comprising any of THCA and CBD, wherein said first (THC) and second cannabinoids are mixed in approximately a n:m ratio by weight. In some embodiments the ratio n:m is approximately 1:1 by weight. A third cannabinoid comprising CBD or THCA, respectively, may also be employed, wherein said first (THC), second, and third cannabinoids are mixed in approximately a n:m:r ratio by weight as described in more detail below.

Another aspect is directed to a method for administering therapeutic formulations to a subject, comprising providing a first quantity of a first cannabinoid comprising THC, providing a second quantity of a second cannabinoid comprising any of THCA and CBD, mixing said first cannabinoid with said second cannabinoid at approximately a n:m ratio, and preparing a dose comprising a formulation of said first and second cannabinoids.

### BRIEF DESCRIPTION OF THE DRAWINGS

For a fuller understanding of the nature and advantages of the present invention, reference is made to the following detailed description of preferred embodiments and in connection with the accompanying drawings, in which:

FIG. 1 depicts a formulation or dose provided to a subject according to the present disclosure; and

FIG. 2 depicts a method, steps or process for providing the formulation above to a subject.

### DETAILED DESCRIPTION

As stated above, cannabinoids have been used to treat or alleviate medical conditions in certain patients and to improve their well-being or physical and mental state. Cannabis-derived cannabinoids in particular have been used for their psychoactive effects, including in dulling the motor nerves to produce a state of relaxation and comfort in its users, e.g., patients with certain medical conditions. THC for example, is found to have an impact on the motor nerves of a subject, which is commonly referred to as a “stoning” effect whereby the subject is relaxed as a result of the THC's effect. However, the present inventor has understood that certain applications of THC and related cannabinoids can be used to reverse this condition and waken, excite or spark the motor nerves of a subject as described below. Specifically, it is found that an optimum combination of cannabinoids can reverse lower motor neuron symptoms in certain subjects, allowing them to regain strength, or to balance the effects of THC against other effects of the present cannabinoids to achieve an improved state of well-being or health in said subjects.

The present inventor, through research, experimentation and analytical methods, has uncovered and optimized previously-unknown formulations and methods for preparing and using the components as described herein, including so as to achieve a reversal of the inhibitory nature of THC and/or other agents on a subject's motor nerves. It is therefore possible and practical to reverse the psychoactive effects of the THC agent or cannabinoid when it is combined with another agent known as cannabidiol (CBD), and/or a third cannabinoid form called tetrahydrocannabinolic acid (THCA) according to the present formulations and techniques.

One formulation according to this disclosure employs controlled ratios of THC and THCA and/or other cannabinoids. An exemplary formulation hereof comprises THCA in combination with THC, in quantities as low as 10 milligrams total, which may be used to stimulate the motor function of the brain. One or more embodiments combine THC and THCA in a ratio of THC:THCA between 1:1 and 1:2. In an example, the formulation comprises THC:THCA combined in proportions being 1:1 plus-or-minus 50%, i.e., where one or the other of the cannabinoids may be up to 50% greater (by volume or by weight) than the other in said formulation. In a preferred analysis, the components are mixed as weight ratios.

Another formulation employs three cannabinoids in a ratio of CBD:THCA:THC. The ratio in a preferred embodiment, found to reverse yet balance the inhibitory effect of THC on a subject's motor nerves and inhibit spasms, comprises substantially similar proportions of CBD, THCA and THC; meaning a formulation comprising approximately CBD:THCA:THC of 1:1:1. By “substantially similar” and “approximately” in this context it is meant that the ratios of the components are within twenty percent (20%) or less of one another. Therefore, without loss of generality, the above example may comprise a ratio of CBD:THCA:THC being, for example, 1.2:1.0:1.1; or 0.85:1.1:1.0; or 1.0:1.0:1.0, etc. Those skilled in the art will appreciate that variations of the particular examples given here are possible, sometimes to suit the needs of a given subject suffering from a given ailment, or may vary depending on the exact chemical nature and source of the cannabinoid being employed, as the subjects, ailments or cannabinoids in one instance can vary somewhat from another.

Yet another formulation hereof comprises a controlled proportion of CBD to THC, which may be combined in approximately an at least six-to-one (6:1) ratio of CBD:THC. In a preferred embodiment, said ratio is approximately eight-to-one, i.e., CBD:THC (8:1) ratio to reverse the effect of THC on the motor nerves. As to dosage, one productized dose may be ingested by a subject in quantities of approximately 10 milligrams (mg) of THC to 80 mg of CBD.

Ratios presented herein should not be considered exact, especially since cannabinoid extractions rarely yield a perfect ratio. An actual ratio that is within plus-or-minus 20% of a ratio stated herein should be considered as contemplated by the disclosure of such ratio herein.

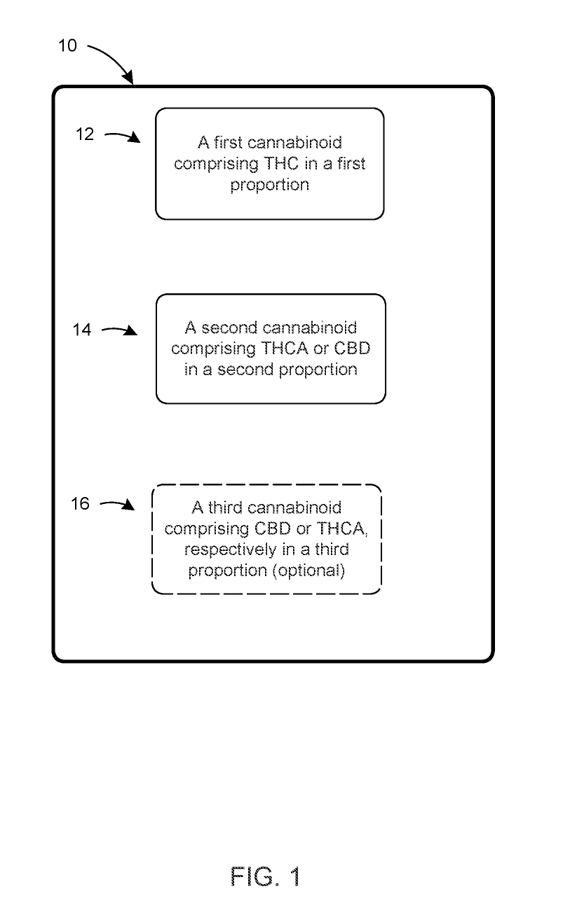

FIG. 1 illustrates an exemplary formulation 10 or dose for administering to a subject comprising a plurality of cannabinoids as discussed above, including a first (THC) cannabinoid 12, a second cannabinoid (THCA or CBD) 14, and optionally a third cannabinoid (CBD or THCA respectively) 16. Each cannabinoid in the dose 10 is provided as disclosed in ratios n:m or n:m:r. The dosage may be ten (10) milligrams of THC in a given formulation and dose product.

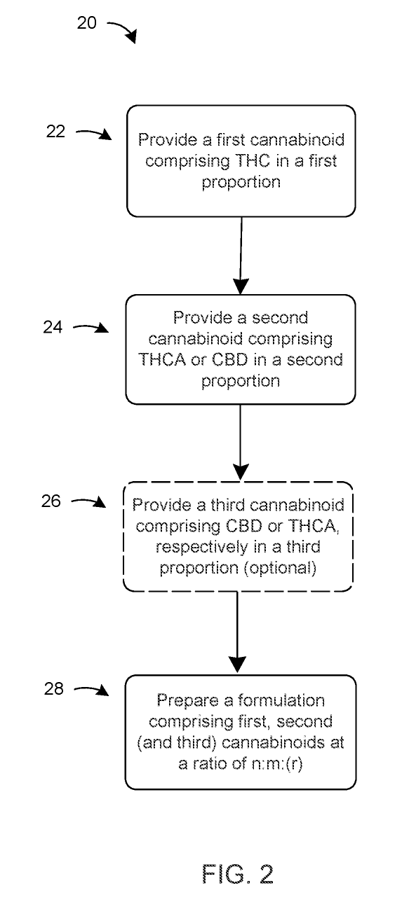

FIG. 2 illustrates an exemplary method or steps or process 20 for providing the formulation above to a subject. The method includes providing a first cannabinoid (THC) at step 22, providing a second (THCA or CBD) cannabinoid at step 24, and optionally providing a third cannabinoid (CBD or THCA, respectively) at step 26. The cannabinoids are mixed as suitable to the administration method and prepared as a formulation or dose to give to a subject.

The preparation can include mechanical or chemical processing steps so as to arrive at a mixture of the cannabinoids in a combustible form so as to give the combustion by-products to the subject, e.g., smoking; or as an ingestible (swallowable, edible) form; or as an injectable form; or as a spreadable topical application.

Those skilled in the art can now appreciate that balancing certain cannabinoids in a formulation can have overall beneficial effects on a subject, including to meter the effects of one cannabinoid alone by including others in useful proportion. The skilled practitioner will also understand that this disclosure can be extended to other or similar cannabinoids without loss of scope. For example, CBN, CBDA or CBC may be metered relative to a dose of THC to achieve benefits in movement, anti-spasm or other symptom improvements or physiological developments. The skilled practitioner will also understand that these formulations and techniques can be combined with complementary practices in medicine, nutrition, exercise and other methods for treating subjects with or even without medical needs, as suitable.

The present formulations and methods may be applied to treatment of persons with a variety of conditions, including neuro-degenerative conditions, as mentioned earlier. In some cases, the present methods and formulations are used in or with the treatment of neuromuscular blocking agents that lead to neurodegenerative diseases and movement (motor) disorders, e.g., Parkinson's disease, ALS, MS, and others.

The present formulations can be incorporated into and manufactured as several compounds and in a variety of form factors, depending on a particular need. In some examples, this can be in a form that is inhaled (smoked) by a patient using a suitable cigarette, pipe, tubing or vessel for delivering smoke from the formulation into the patient's lungs. In another example, the formulation is prepared as an ingestible product, e.g., in a stand-alone food, beverage, or incorporated into a known food or beverage product for ingestion, or incorporated into a sublingual spray. In yet another example, the formulation is prepared as a topical product, cream, etc., which can be applied to a patient trans-dermally using direct application to the patient's skin, or in a patch applied to the patient at a selected region of the patient's body. And in still another example, the formulation can be delivered directly into the patient's bloodstream using an intravenous drip, intramuscular or subcutaneous injection or similar means.

Some cannabinoids are obtained through development of “strains” deriving from the marijuana plant or other natural or synthetic sources. A strain known as sativa-dominant may be used to target bulbar symptoms of a subject in some instances. In other instances, an indica-dominant strain may be used to target the limbs of a patient.

With regard to the above formulations, they may be included in a more robust compound or product as a subset of the ingredients of said compound or product. Further agents and ingredients that may be present in said more robust compound or product can include: fat binding agents, e.g., sublingual coconut, hemp, or emu oils, tinctures or butters; transdermal delivery mechanisms such as patches, as mentioned above; or topically in the form of lotions, creams, balms, ointments, or gels applied to the skin of a patient.

Those skilled in the art will appreciate that the present disclosure of formulations and methods for delivering the same can be applied to a wide variety of conditions, which specific applications will depend on the needs and condition of a given patient. It is not intended that any person self-diagnose or self-administer or manufacture any product, nor administer any such product, if these actions are contrary to the law or medical advice by a qualified professional. This disclosure is merely to describe the present inventor's discovery of the types of formulations, in certain proportions to one another, and potential applications of the same where appropriate. The response and sensitivity of any individual may vary according to numerous factors, which should be taken into consideration by the individual and his or her medical practitioner prior to making or using the present formulations or methods.

What is claimed is:

1. A therapeutic formulation of cannabinoids, comprising:
a first cannabinoid comprising THC;
a second cannabinoid comprising any of THCA and CBD;
wherein said first (THC) and second cannabinoids are mixed in approximately a n:m ratio by weight.

2. The formulation of claim 1, wherein said approximately a n:m ratio by weight comprises an approximately 1:1 ratio by weight.

3. The formulation of claim 2, wherein said first and second cannabinoids are mixed to within twenty (20) percent (%) of one another by weight.

4. The formulation of claim 1, wherein said second cannabinoid comprises THCA.

5. The formulation of claim 1, wherein said second cannabinoid comprises CBD.

6. The formulation of claim 4, comprising a dose unit including approximately 10 milligrams of a first (THC) cannabinoid and approximately 10 milligrams of a second THCA cannabinoid.

7. The formulation of claim 1, wherein said approximately a n:m ratio by weight comprises an approximately 1:8 ratio by weight.

8. The formulation of claim 7, comprising a dose unit including approximately 10 milligrams of a first (THC) cannabinoid and approximately 80 milligrams of a second CBD cannabinoid.

9. The formulation of claim 1, further comprising a third cannabinoid, wherein said first (THC), second, and third cannabinoids are mixed in approximately a n:m:r ratio by weight.

10. The formulation of claim 9, wherein the ratio n:m:r of the three cannabinoids (THC:THCA:CBD) is approximately 1:1:1.

11. The formulation of claim 10, comprising a dose unit including approximately 10 milligrams of said first (THC) cannabinoid and approximately 10 milligrams of said second (THCA) cannabinoid and approximately 10 milligrams of said third (CBD) cannabinoid.

12. The formulation of claim 7, comprising a dose unit including approximately 10 milligrams of said first (THC) cannabinoid and approximately 10 milligrams of said second (THCA) cannabinoid and approximately 80 milligrams of said third (CBD) cannabinoid.

13. The formulation of claim 1, wherein said cannabinoids are provided in a combustible mixture.

14. The formulation of claim 1, wherein said cannabinoids are provided in an ingestible mixture.

15. The formulation of claim 1, wherein said cannabinoids are provided in an injectable mixture.

16. The formulation of claim 1, wherein said cannabinoids are provided in a topical spread mixture.

17. A method for administering therapeutic formulations to a subject, comprising:
providing a first quantity of a first cannabinoid comprising THC;
providing a second quantity of a second cannabinoid comprising any of THCA and CBD;
mixing said first cannabinoid with said second cannabinoid at approximately a n:m ratio; and
preparing a dose comprising a formulation of said first and second cannabinoids.

18. The method of claim 17, further comprising:
providing a third quantity of a third cannabinoid wherein said third cannabinoid comprises any of CBD and THCA, respectively, in approximately a n:m:r ratio, and wherein said third cannabinoid is not the same as said second cannabinoid, insofar as: if said second cannabinoid comprises THCA then said third cannabinoid comprises CBD, and if said second cannabinoid comprises CBD then said third cannabinoid comprises THCA.
